(19) United States
(12) Patent Application Publication
(10) Pub. No.: US 2026/0203709 A1
(43) Pub. Date: Jul. 16, 2026

(54) SYSTEM AND METHOD FOR MANAGING INVENTORY OF BULKY PRODUCTS STORED ON SHELVES WITH RFID TAGS

(71) Applicant: WAVEMARK, INC., Concord, MA (US)

(72) Inventors: Adriano C. APOSTOLICO, Holliston, MA (US); Leo KIEFER, Westford, MA (US); Brent E. KOEPPEL, Natick, MA (US); Gregory J. LYON, North Reading, MA (US); Andrew P. WOJTOWICZ, Somerville, MA (US)

(21) Appl. No.: 19/446,485

(22) Filed: Jan. 12, 2026

Related U.S. Application Data
(60) Provisional application No. 63/744,616, filed on Jan. 13, 2025.

(51) Int. Cl. G06Q 10/087; G06K 7/10; G06K 19/07; G16H 40/20

(52) U.S. Cl. CPC G06Q 10/08726; G06K 7/10297; G06K 19/0723; G06Q 10/08724; G16H 40/20

(57) ABSTRACT

Disclosed herein are computer-implemented systems and methods for managing inventory of bulky products stored on a shelving system. The system includes an RFID antenna disposed at a designated location, and RFID reader to detect RFID tags disposed at the designated location, and transmit messages indicating the detections. The antenna is for communicating with RFID tags disposed proximate to the antenna. In one aspect, a pair of RFID tagged cards are inserted in a housing module affixed to the shelving system where bulky products are stored. When a predetermined quantity of the product is removed, an RFID tagged card associated with the product is placed at the designated location. The RFID reader transmits messages to a computing device indicating detections of tags. The computing device determines when the number of times read during a tag detection time period is at least two, and generates a message for replenishing the respective product.

### CROSS-REFERENCE TO RELATED APPLICATION

This application claims priority to U.S. Provisional Patent Application No. 63/744,616, filed January 13, 2025, which is hereby incorporated by reference in its entirety.

### FIELD OF TECHNOLOGY

Aspects of the present disclosure relate to technology in inventory management in a hospital environment.

### BACKGROUND

Repetitive product cycle counts and ordering processes can slow down hospital staff and reduce supply chain efficiency of inventory management systems. Often, staff members need to make storeroom visits and verify inventory to ensure replenishment of consumable products is timely. For example, the inventory management system for a hospital may be a two-bin Kanban system where each product is assigned to one of two bins, a first bin for active inventory of the product and a second bin for reserve inventory of the product. Thus, the first bin is for storing half of the par quantities of a small product for immediate use, while the second bin is for storing half of the par quantities of the small product that will be needed in the near future. The small products stored in the bins may not be individually affixed with a tracking means.

In addition, some hospitals have started using a special type of shelving system referred to as an “Auto Order Shelf” which may be used in conjunction with the two-bin Kanban system. The Auto Order Shelves are used for physically small products placed in Radio Frequency Identification (RFID) tagged bins, such as the two bins of the Kanban system described above. Each bin of the Kanban system is RFID tagged and includes a specific small product. The Auto Order Shelves include a designated location proximate to an RFID reader. When an RFID tagged bin is placed at the designated location, e.g., top shelf, the RFID reader reads the RFID tag of the bin. The inventory management system associates the tags of the bins with the specific products placed in the respective bins. Thus, when a bin is placed at the designated location, the RFID tag is read, and the inventory management system becomes aware of which products are consumed from the bin.

For example, once the quantity of items in a bin is consumed, the empty bin needs to be replenished. The Auto Order Shelf signals that a given bin needs to be replenished when the RFID tagged bin is placed onto the Auto Order Shelf which, via a built-in RFID reader, reads the tag of the bin, identifies the product associated with that bin, and initiates the process for placing an order for a predetermined quantity of the given product in order to replenish that bin.

In some scenarios, orders may be automatically triggered to reduce time needed to replenish supplies. In other scenarios, an alert is sent to inventory managers for reviewing and triggering orders. The automation reduces the chance for running out of stock of consumable products.

Thus, the Auto Order Shelves allow product procurement and replenishment to be automated – thereby reducing burden on hospital staff. The automated process is intended to be integrated into existing clinical workflows. The process for using the Auto Order Shelves is not expected to require extra training of hospital staff beyond the training during a normal orientation stage for new staff. Staff members simply need to place the RFID tagged bins at the designated location. However, the tagged bins are not suitable for bulky products.

Therefore, there is a need for a shelving system and method that provides an efficient supply chain management for bulky products used in hospital environments as these products do not fit in bins.

### SUMMARY

In one aspect, the pair of RFID tagged cards are further tagged with respective human readable labels and/or machine readable barcodes. In addition, a PAR level is established for the product. The PAR level corresponds to a predetermined quantity of the product that corresponds to a single pair of RFID tagged cards. The pair of RFID cards are individually removable from the housing module by a staff member. When a predetermined quantity of the product associated with an RFID tagged card is used, a staff member removes one RFID tagged card from the housing module and places the removed RFID tagged card at a designated location proximate to an RFID reader. For each read RFID tag, the RFID reader transmits a message to a computing device of the PAR system, the message including an identifier of the removed RFID tagged card. The computing device receives and processes messages from the RFID reader and places orders for replenishment of the bulky products, as appropriate.

In particular, the present disclosure provides a method for managing an inventory of bulky products stored on a shelving system suitable for storing the bulky products, the shelving system including an RFID antenna disposed adjacent a designated location and an RFID reader in communication with the RFID antenna and at least one computing device, the RFID reader configured to perform scans via the RFID antenna to detect RFID tags disposed at the designated location on the shelving system, the method comprising: inserting a pair of RFID tagged cards in a housing module affixed to storage shelf(s) on which such bulky products are stored and, with each RFID tagged card having a distinct respective RFID tag, the pair of RFID tagged cards in the housing module being associated with a single bulky product, and a PAR level being established for the bulky product; when a predetermined quantity of the bulky product is removed from the shelving system, placing an RFID tagged card associated with the bulky product at the designated location on the shelving system, the predetermined quantity being a proportion based on the PAR level; by the RFID reader, performing a scan to detect the RFID tag disposed at the designated location, and transmit a message to the at least one computing device indicating detection of the RFID tag; by the at least one computing device, receiving the message from the RFID reader indicating detection of the RFID tag placed at the designated location, tracking a number of times the RFID tag is read during each tag detection time period, deduplicating received messages for the detected RFID tag when applicable, the deduplicating including consolidating multiple detections of the same RFID tag within a tag detection time period, and, when the number of times the detected RFID tag is read during the tag detection time period is at least two, generating a message for replenishing the bulky product associated with the RFID tagged card based on the PAR level; and transmitting the generated message for replenishment of the bulky product to an automated ordering system or staff.

The present disclosure also encompasses a system and a computer program product that implement this method, allowing for seamless integration of the method for managing inventory of bulky products stored on a shelving system into existing inventory systems used for smaller items.

In one example aspect, an apparatus for managing an inventory of bulky products stored on a shelving system is provided, the apparatus comprising: an RFID antenna disposed at a designated location on a shelving system suitable for storing the bulky products, the RFID antenna configured to communicate with RFID tags disposed proximate to the RFID antenna; an RFID reader including the RFID antenna, the RFID reader configured to communicate with at least one computing device, the RFID reader configured to control the antenna to scan and detect RFID tags disposed at the designated location on the shelving system, and to transmit messages to the at least one computing device, the messages indicating detections of RFID tags; and a housing module storing a pair of RFID tagged cards, the housing module being affixed to a storage fixture of the shelving system on which bulky products are stored, each RFID tagged card having a distinct respective RFID tag, the pair of RFID tagged cards in a same housing module being associated with a single bulky product, and a PAR level being established for the bulky product, wherein the at least one computing device is configured to: receive messages from the RFID reader indicating detections of RFID tags when the RFID tagged cards are placed at the designated location on the shelving system; for each detected RFID tag, track a number of times the RFID tag is read during each tag detection time period, deduplicate received messages when applicable, and when the number of times the RFID tag is read during the tag detection time period is at least two, generate a message for replenishing the bulky product associated with the RFID tagged card, wherein a quantity of the replenishment is based on the PAR level established for the bulky product.

In one example aspect, the RFID tagged cards include human readable labels and/or machine readable barcodes.

In one example aspect, the PAR level corresponds to a predetermined quantity of the bulky product that corresponds to a single pair of RFID tagged cards.

In one example aspect, the pair of RFID cards are individually removable from the housing module.

In one example aspect, the pair of RFID tagged cards placed in a same housing module are interchangeable in that they are insertable in either of two slots available per housing module.

In one example aspect, a shape of the RFID tagged card corresponds to a shape of a slot of the housing module such that the tagged card can only be inserted in a single orientation into the slot of the housing module.

According to one example aspect of the disclosure, a computer-implemented system is provided for managing inventory of bulky products stored on a shelving system, the shelving system including an RFID antenna disposed adjacent a designated location and an RFID reader in communication with the RFID antenna and at least one computing device, the RFID reader configured to perform scans via the RFID antenna to detect RFID tags disposed at the designated location on the shelving system, the computer-implemented system comprising: at least one memory; and at least one hardware processor coupled with the at least one memory and configured, individually or in combination, to: control the RFID reader to perform a scan to detect at least one RFID tag disposed at the designated location, wherein the at least one RFID tag is from a pair of RFID tagged cards stored in a housing module affixed to a storage fixture of the shelving system on which bulky products are stored, with each RFID tagged card having a distinct respective RFID tag, the pair of RFID tagged cards in the housing module being associated with a single bulky product, and a PAR level being established for the bulky product, wherein when a predetermined quantity of the bulky product is removed from the shelving system, an RFID tagged card associated with the bulky product is placed at the designated location on the shelving system, the predetermined quantity being a proportion based on the PAR level; control the RFID reader to transmit a message to the at least one computing device indicating detection of the RFID tag; by the at least one computing device, receive the message from the RFID reader indicating detection of the RFID tag placed at the designated location, track a number of times the RFID tag is read during each tag detection time period, deduplicate received messages for the detected RFID tag when applicable, when the number of times the detected RFID tag is read during the tag detection time period is at least two, generate a message for replenishing the bulky product associated with the RFID tagged card based on the PAR level; and transmit the generated message for replenishment of the bulky product to an automated ordering system or staff

In one example aspect, a non-transitory computer-readable medium storing a set of instructions thereon for managing inventory of bulky products stored on a shelving system suitable for storing the bulky products, the shelving system including an RFID antenna disposed adjacent a designated location and an RFID reader in communication with the RFID antenna and at least one computing device, the RFID reader configured to perform scans via the RFID antenna to detect RFID tags disposed at the designated location on the shelving system, wherein the set of instructions comprises instructions for: controlling the RFID reader to perform a scan to detect at least one RFID tag disposed at the designated location, wherein the at least one RFID tag is from a pair of RFID tagged cards stored in a housing module affixed to a storage fixture of the shelving system on which bulky products are stored, with each RFID tagged card having a distinct respective RFID tag, the pair of RFID tagged cards in the housing module being associated with a single bulky product, and a PAR level being established for the bulky product, wherein when a predetermined quantity of the bulky product is removed from the shelving system, an RFID tagged card associated with the bulky product is placed at the designated location on the shelving system, the predetermined quantity being a proportion based on the PAR level; controlling the RFID reader to transmit a message to the at least one computing device indicating detection of the RFID tag; by the at least one computing device, receiving the message from the RFID reader indicating detection of the RFID tag placed at the designated location, tracking a number of times the RFID tag is read during each tag detection time period, deduplicating received messages for the detected RFID tag when applicable, the deduplicating including consolidating multiple detections of the same RFID tag within a tag detection time period, when the number of times the detected RFID tag is read during the tag detection time period is at least two, generating a message for replenishing the bulky product associated with the RFID tagged card based on the PAR level; and transmitting the generated message for replenishment of the bulky product to an automated ordering system or staff.

### BRIEF DESCRIPTION OF THE DRAWINGS

The accompanying drawings, which are incorporated into and constitute a part of this specification, illustrate one or more example aspects of the present disclosure and, together with

the detailed description, serve to explain their principles and implementations. The foregoing and other objects, features, and advantages will be apparent from the following more detailed description of exemplary aspects, as illustrated in the accompanying drawings. In the drawings, like reference characters generally refer to the same parts throughout the different views. The drawings are provided for illustrative purposes and are not necessarily to scale, with emphasis instead placed on illustrating the principles of the various aspects.

FIG. 1 illustrates a high-level block diagram of a system for managing inventory of bulky products stored on a shelving system in accordance with aspects of the present disclosure.

FIG. 2 provides an example schematic of a housing module in accordance with aspects of the present disclosure.

FIG. 3 provides an example schematic of a card B in accordance with aspects of the present disclosure.

FIG. 4 presents a flowchart of a method for managing inventory of bulky products stored on a shelving system in accordance with aspects of the present disclosure.

FIG. 5 is a block diagram illustrating various components of an example computer system via which aspects of the present disclosure may be implemented.

FIG. 6 is a block diagram of various example system components, usable in accordance with aspects of the present disclosure

FIG. 7A provides an example schematic of another implementation of a card B in accordance with aspects of the present disclosure.

FIG. 7B provides an example schematic of a electronic shelf label bracket in accordance with aspects of the present disclosure.

FIG. 8A provides an example schematic of an implementation of a card B within a housing module in accordance with aspects of the present disclosure.

FIG. 8B provides an example schematic of another implementation of a card B within a housing module in accordance with aspects of the present disclosure.

### DETAILED DESCRIPTION

Example aspects are described herein in the context of an apparatus, system, method, and various computer program features for managing inventory of bulky products stored on a shelving system.

In one aspect, the method uses a Periodic Automatic Replenishment (PAR) system and a housing module affixed to the storage shelf(s) on which such bulky products are stored and designed to house a pair of RFID tagged cards with each RFID tagged card having its own respective RFID tag. The pair of RFID tagged cards in a same housing module are associated with a single replenishable product. In one aspect, the pair of RFID tagged cards are further tagged with respective human readable labels and/or machine readable barcodes. In addition, a PAR level is established for the product. The PAR level corresponds to a predetermined quantity of the product that corresponds to a single pair of RFID tagged cards. The pair of RFID cards are individually removable from the housing module by a staff member. When a predetermined quantity of the product associated with an RFID tagged card is used, a staff member removes one RFID tagged card from the housing module and places the removed RFID tagged card at a designated location proximate to an RFID reader. For each read RFID tag, the RFID reader transmits a message to a computing device of the PAR system, wherein the message includes the RFID identifier associated with the product.

The computing device of the PAR system receives messages from RFID readers, processes the received messages tracking a number of times each particular tag is read during each tag detection time period, and deduplicates received messages for each particular tag when applicable. The computing device of the PAR system determines whether or not the particular RFID tag is detected at least twice in the tag detection time period. When the particular RFID tag is detected at least twice in the tag detection time period, the computing device of the PAR system generates a message for replenishing the product associated with the particular RFID tagged card. The generated message may be used for automatic orders and/or for sending alerts to staff members tasked with verifying and placing orders or tasked with replenishing products. If the same tag is detected after the message for ordering has already been sent, the PAR system is aware that the order has already been placed. However, if the RFID tag of the second card in the same housing is detected, the PAR system generates a new message, i.e., a second message, for further replenishment of the product.

As described above, the computing device deduplicates received messages for each particular tag when applicable. This step is necessary in order to prevent duplicate orders if the same RFID tagged card is read and reported by the RFID reader more than twice during the reading-reporting period. For example, if a card is temporarily removed from the reader, then replaced on the reader, a duplicate message would be received. This may occur, for example, if a staff member is inspecting which cards are on the reader. The requirement for reading the RFID tagged card twice during the reporting period is to ensure that orders are not placed when staff members temporarily place RFID tagged cards within the read range of the RFID antenna-reader. This may occur during restocking or any other times when staff members are handling products near or on the shelf at which products are stored before use.

In one aspect, the deduplication is performed at a predetermined time. For example, a hospital may establish a cutoff time for placing orders for replenishment of products. When the cutoff time is reached, orders may be bundled into an electronic order and sent to a supply chain person to review and submit the order to the supplier. The cutoff times may be different for different supplies, different suppliers, different hospitals, etc. Therefore, the deduplication may be coordinated in time to facilitate ordering of appropriate quantities at the respective cutoff times.

In one aspect, if both RFID tagged cards associated with a product are detected, the method may generate a further message indicating that both tags are detected. The further message indicating that both tags are detected, the quantity of product is completely depleted and requires replenishment of the full quantity of product. By analyzing the frequency of these messages over extended periods of time, the system may generate notifications that may be sent to staff members and/or systems to adjust the appropriate PAR level for the product. For example, the PAR level may be increased to correspond to appropriate inventory level for the product. In contrast, if only one card is read for a product for an extended time period, e.g., a year, it may indicate a necessity to reduce the PAR level.

In one aspect, the feature for setting the appropriate PAR level is integrated into systems that track expiration dates of products to ensure proper inventory levels for consumption prior to expiration, or to indicate product is expiring or expired on the shelf and adjust PAR levels in order to minimize product expiration.

In one aspect, the housing module of the present disclosure may be used in conjunction

with the Auto Order Shelf described above. That is, in lieu of placing tagged bins in the designated location of the Auto Order Shelf, for bulky items, the RFID tagged cards are removed and placed at the same location where the same RFID antenna-reader can read the RFID tagged cards and the RFID tagged bins.

Further, as used herein, “shelf” or “shelving system” encompasses any storage fixture or receptacle suitable for holding bulky products, including bins, carts, cabinets, racks, wall mounts, pegboards, mobile trolleys, and modular frames. References to a “shelf” are therefore non-limiting and apply equally to such storage fixtures.

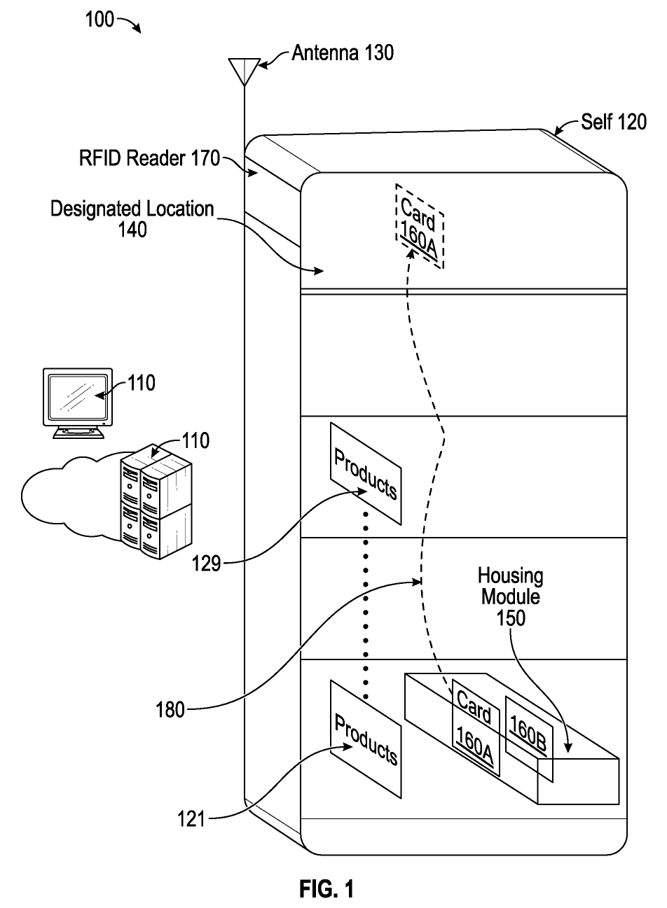

FIG. 1 illustrates a high-level block diagram of a system 100 for managing inventory of bulky products stored on a shelving system in accordance with aspects of the present disclosure. The system 100 includes at least one computing device 110 with memory and at least one processor, at least a shelf 120 for storing bulky products, an RFID antenna 130 disposed at a designated location 140 on the at least one shelf 120. The RFID antenna 130 is used for communicating with RFID tags 160A and 160B disposed proximate to the RFID antenna 130. The system 100 further comprises any number of a housing module 150 with each housing module 150 storing a pair of RFID tagged cards 160A and 160B. Each RFID tagged card 160A or 160B has a distinct respective RFID tag. The pair of RFID tagged cards 160A and 160B in a same housing module 150 are associated with a single bulky product, and a PAR level is established for the bulky product by a user. The system 100 further includes an RFID reader 170 in communication with the RFID antenna 130 and at least one computing device 110. The RFID reader 170 is configurable for performing scans (via the RFID antenna 130) to detect RFID tags 160A, 160B disposed at the designated location 140 on the shelf 120, and transmit messages to the at least one computing device 110, wherein the messages indicate detections of particular RFID tags. The at least one computing device 110 receive messages from the RFID reader 170 indicating detections of RFID tags 160A, 160B when the RFID tagged cards are placed at the designated location 140. In addition, for each detected RFID tag 160A or 160B, the at least one computing device 110 tracks a number of times the RFID tag is read during each tag detection time period, and deduplicates received messages when applicable. Then, when the number of times the RFID tag 160A or 160B is read during the tag detection time period is at least two, the at least one computing device 110 generates a message for replenishing the bulky product associated with the RFID tagged card. The quantity of the replenishment is based on the PAR level established for the bulky product.

In one aspect, when there are two RFID tagged cards, e.g., 160A and 160B, in a single housing module 150, the predetermined quantity corresponds to 50% of the PAR level established for the product associated with the card. For an illustrative example, suppose a housing module 150 includes a pair of RFID tagged cards labeled card A and card B (e.g., 160A and 160B). Both card A and card B of the housing module 150 are associated with a bulky product. The user selects which bulky product is to be associated with the given pair of RFID tagged cards. For instance, the bulky product may be selected from: scrubs 121, socks 122, gowns 123, sheets 124, blankets 125, hospital pitchers 126, canes 127 (may be adjustable), crutches 128, instrument trays 129, and the like. Suppose the selected bulky product is a scrub 121 and the PAR level is ten. Then, card 160 A may be removed and placed at the designated location 140 when five scrubs are removed from the shelf, as shown in 180. The RFID reader 170 reads the RFID tag of card 160 A and generates a product replenishment message for the five scrubs 121. For the purpose of human readability, card 160 A may be labeled as “A” and/or “scrubs” and card 160 B may be labeled as “B” and/or “scrubs”. The labels are placed on the RFID tagged cards such that they are readily visible during handling of the cards by staff members, e.g., during inserting and removal from the housing module.

In one aspect, the pair of RFID tagged cards 160A, 160B placed in a same housing module 150 are interchangeable in that they can be inserted in either of two slots available per housing module. In addition, while the pair of cards are interchangeable, in order to simplify ease of use and comply with the A-B convention practiced in the two-bin kanban system, the orientation of the receiving slot in the housing inherently positions the first (front) card as the “A” card and the second (rear) card as the “B” card. Furthermore, the computing system may use logic during the reading-reporting period to determine whether the identified card corresponds to the first (“A”) order or the second (“B”) order to be placed for the product. Therefore, the cards are interchangeable, yet become directional when inserted into housing – thereby automatically creating an A/B Kanban inventory management system for bulky products. Similar to the system used for smaller products, the system of the present disclosure visually displays the status of inventory level when viewing the cards in the housing and logically displays the status using the RFID reader & computing system to associate the card detection during the reading-reporting

period with the A/B convention of the two-bin Kanban system. The system of the present disclosure supports FIFO product utilization and indicates urgency of need for replenishment. In addition, the RFID tagged cards are compatible with the Auto Order Shelf (reader-antenna shelf unit), and therefore the system of the present disclosure does not require a “separate” reader-antenna. As such, the system can be integrated with conventional two-bin storage systems using an Auto Order Shelf.

In one aspect, the shape of the RFID tagged card 160A, 160B is designed in a manner a human can readily determine an orientation for inserting the card into a given slot of the housing module 150.

In another aspect, the housing module 150 may further include one or more flanges covering RFID tagged cards 160A, 160B placed in the housing module 150. In another aspect, the RFID tagged cards 160A, 160B include one or more flanges for covering the opposing RFID tagged cards 160A, 160B placed in the housing module 150.

In one aspect, when a number of flanges is two or more, the flanges are placed such that a first flange of the one or more flanges covering a first RFID tagged card 160A sits on top of a second flange of the one or more flanges covering a second RFID tagged card 160B such that the second RFID tagged card 160B can be disengaged from the housing module 150 only after the first RFID tagged card 160A is disengaged.

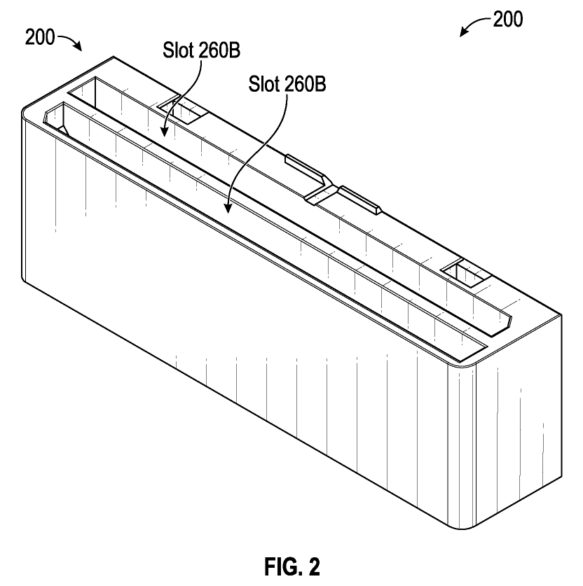

FIG. 2 provides an example schematic 200 of a housing module, e.g., the housing module 150 described above in accordance with aspects of the present disclosure. The housing module includes two slots for cards. For the example scenario, the two slots 260A and 260B are to be used for two RFID tagged cards, such as the cards 160A and 160B shown in FIG. 1.

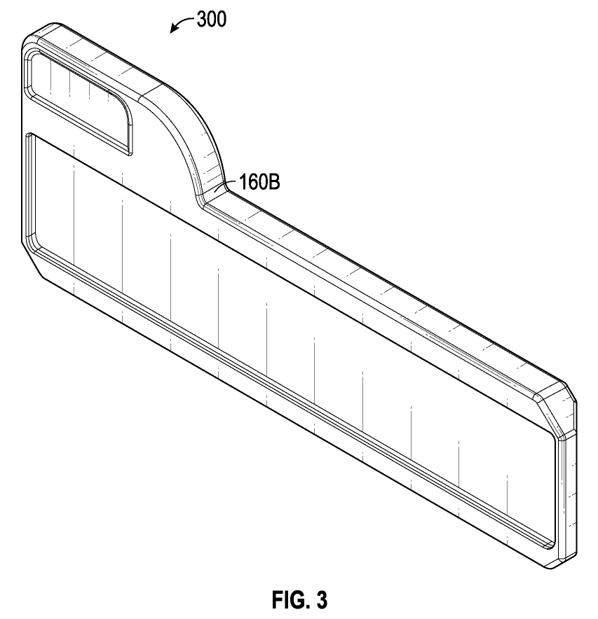

FIG. 3 provides an example schematic 300 of a card B, e.g., card 160B, in accordance with aspects of the present disclosure. The shapes of the cards are such that each card is insertable into the housing module in either slot but in a different orientation. The label on the card, A or B, is visible to a staff member viewing the product and moving the card to the designated location on the shelf when the product associated with the card is adequately depleted, such as when it is half depleted for card A, or completely depleted for card B.

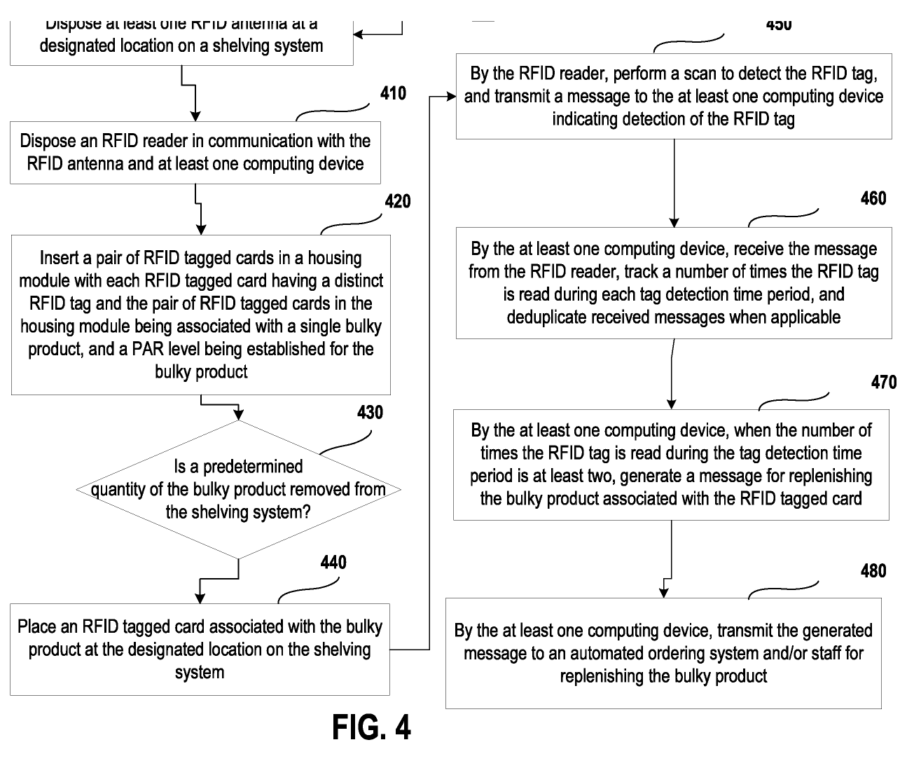

FIG. 4 presents a flowchart that illustrates a method 400 for managing inventory of bulky products stored on a shelving system in accordance with aspects of the present disclosure.

Although method 400 may be implemented for a hospital environment, the method may be implemented for other fast paced environments in which accuracy and proper stocking of bulky products and inventory level is critical. Thus, the following detailed description outlines each step of method 400, along with its rationale and potential variations in implementation of the particular step without further limiting the method of the present disclosure. Although the foregoing examples reference a hospital environment, method 400 is equally applicable to other environments where accurate stocking of bulky products is significant, including but not limited to manufacturing, emergency services, retail backrooms, warehousing etc..

Method 400 starts in step 402 and proceeds to step 405.

In step 405, method 400 includes disposing at least one RFID antenna at a designated location on a shelving system suitable for storing the bulky products. The RFID antenna is used for communicating with RFID tags disposed proximate to the RFID antenna. For example, the RFID tags associated with the product 121 are read when tag 160A or 160B shown in FIG. 1 is moved to the designated location 140.

In step 410, method 400 includes disposing an RFID reader in communication with the RFID antenna and at least one computing device. For example, an RFID reader 170 is disposed in communication with the RFID antenna 130 and at least one computing device 110. The RFID reader is used for performing scans to detect RFID tags, e.g., RFID tag of RFID tagged card 160A after it has been moved as illustrated in 180, disposed at the designated location 140 on the shelf 120, and transmitting messages to the at least one computing device 110. The messages indicate detections of RFID tags. In some implementations, the RFID tags may be encoded in accordance with RAIN/GS1 schemes such that pull‑card tags are distinguished from non‑pull‑card tags. The RAIN/GS1 encoding schemes enable RFID readers to filter tags based on EPC prefixes, user‑memory flags, or custom command sets to avoid reporting non‑pull‑card tags, and to prevent non‑authorized readers from reporting pull‑card tags.

According to one aspect of the disclosure, while RFID implementations are described, alternative detection modalities may be used, including passive or low‑energy Bluetooth beacons, optical codes (e.g., QR/Datamatrix) read by cameras, magnetic or Hall sensors, mechanical switches, or computer‑vision detection of card presence/absence, any of which may be used to trigger replenishment workflows and optional display updates.

In step 420, method 400 includes inserting a pair of RFID tagged cards in a housing module 150, with each RFID tagged card having a distinct respective RFID tag (e.g., 160A and 160B). The pair of RFID tagged cards in the housing module 150 are associated with a single bulky product, e.g., one of products 121, 122, …, 129, and a PAR level is established for the bulky product. In some implementations, one, two, or more RFID tagged cards may be associated with a given item, with each card corresponding to a distinct inventory threshold or workflow state (e.g., partial depletion, reorder point, stockout).

In further implementations, the pair of RFID tagged cards may be assigned to respective slots of the housing module (e.g., a first card in slot 260A and a second card in slot 260B), each having a distinct RFID identifier corresponding to different inventory thresholds (e.g., a first threshold approximating half of the PAR level and a second threshold approaching full depletion). Placement of the first card at the designated location (e.g., by removal from slot 260A) may prompt a first replenishment or review action, and placement of the second card (e.g., by removal from slot 260B) may prompt an escalated replenishment action.

In step 430, method 400 includes human monitoring by the user to determine when a predetermined quantity of the bulky product is removed from the shelving system. The predetermined quantity is a proportion based on the PAR level. For example, the predetermined quantity may be half of the PAR level for the product. When the predetermined quantity of the bulky product is determined as being removed from the shelving system, the method proceeds to step 440. Otherwise, the method remains in step 430 and continues to monitor. In some implementations, cards and/or pull tabs may be color‑coded (e.g., red‑yellow‑green) or patterned to visually communicate inventory status corresponding to depletion thresholds, which may supplement or operate independently of electronic detection.

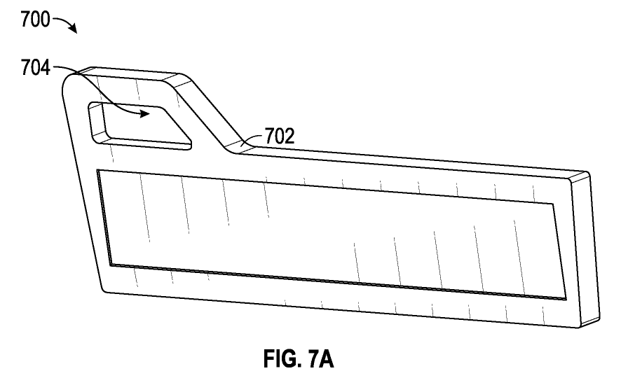

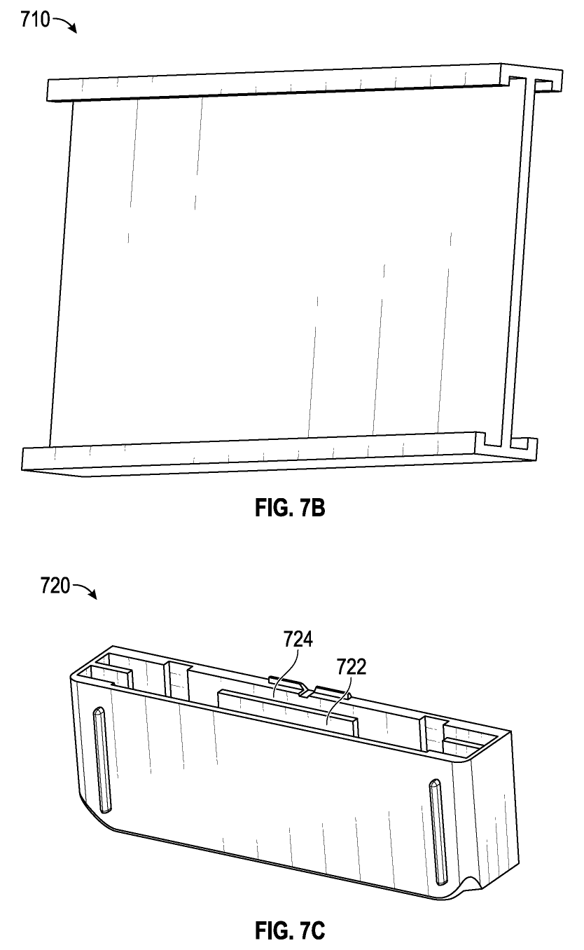

In some implementations, the RFID tagged card may be removably disposed within a housing module having multiple slots, such as slots 260A and 260B (which may be analogous to slots 722 and 724 of FIG. 7C), and the card includes a pull tab with a through-hole through which a tether is routed to physically retain the card to the housing module, as described below. For example, the tether permits sufficient range of motion for the user to remove the card from the housing module, present the card to the RFID antenna for reading, and return the card to the appropriate slot, while reducing the risk of card misplacement or loss.

In step 440, method 400 includes placing an RFID tagged card associated with the bulky product at the designated location on the shelving system.

In step 450, method 400 includes performing, by the RFID reader, a scan to detect the RFID tag disposed at the designated location, and transmits a message to the at least one computing device indicating detection of the RFID tag. For example, a scan may be performed to detect RFID tag of 160A. The detected RFID tag is transmitted to the computing device 110. In further implementations, the housing module may include one or more sensors (e.g., mechanical, optical, or magnetic) or indicators (e.g., LEDs) to detect and/or signal card-in-slot status, proper placement, or successful reader detection of the RFID tag. For example, the computing device may drive visual or audible cues to guide the user during removal, reading, and return operations, thereby improving detection reliability and workflow compliance.

In step 460, method 400 includes receiving, by the at least one computing device, the message from the RFID reader indicating detection of the RFID tag placed at the designated location. For example, the computing device 110 receives the message from the RFID reader 170 indicating detection of the RFID tag of card 160A placed at the designated location 140. The computing device 110 tracks a number of times the RFID of card 160A is read during each tag detection time period, and deduplicates received messages for the detected RFID tag of card 160A, when applicable. The “tag detection time period” may be a configurable interval during which multiple identical tag reads are aggregated to mitigate transient or duplicate detections arising from user movement or RF reflections. Within the tag detection time period, the computing device deduplicates repeated detections of the same tag and applies a debounce rule (e.g., requiring threshold multiple reads) before confirming a valid placement event. In some implementations, a return of the card to the housing module (e.g., detected by cessation of reads for at least a timeout or by a sensor) resets the detection state for subsequent events. Moreover, the computing device may apply a retention window during which a temporary removal and re‑presentation of the same RFID tag does not constitute a new detection event unless a prescribed retention period is exceeded. If the card is re‑presented within the retention window, the event is treated as a continuation of the prior detection rather than a new replenishment trigger.

In step 470, method 400, includes generating, by the at least one computing device, when the number of times the detected RFID tag is read during the tag detection time period is at

least two, a message for replenishing the bulky product associated with the RFID tagged card based on the PAR level. For example, the computing device 110 determines when the RFID tag of card 160A is read at least two times within the tag detection time period and generates a message for replenishing the bulky product 121 which is associated with the RFID tagged card 160A.

In some implementations, method 400 further includes, responsive to generating the message for replenishment and/or a change in PAR level, transmitting display update content to an electronic shelf label (ESL) (which may refer to an electronic storage fixture label) associated with the bulky product (e.g., using electronic shelf label bracket 710). The computing device formats content (e.g., updated PAR, on-hand estimate, bin location, status) and communicates the content via one or more networks and intermediary servers to the ESL for wireless (e.g., Wi-Fi) display. In implementations without an ESL, the method relies on a paper label on the front face of the housing to present static item information, while replenishment messages continue to be transmitted to staff and/or an ordering system.

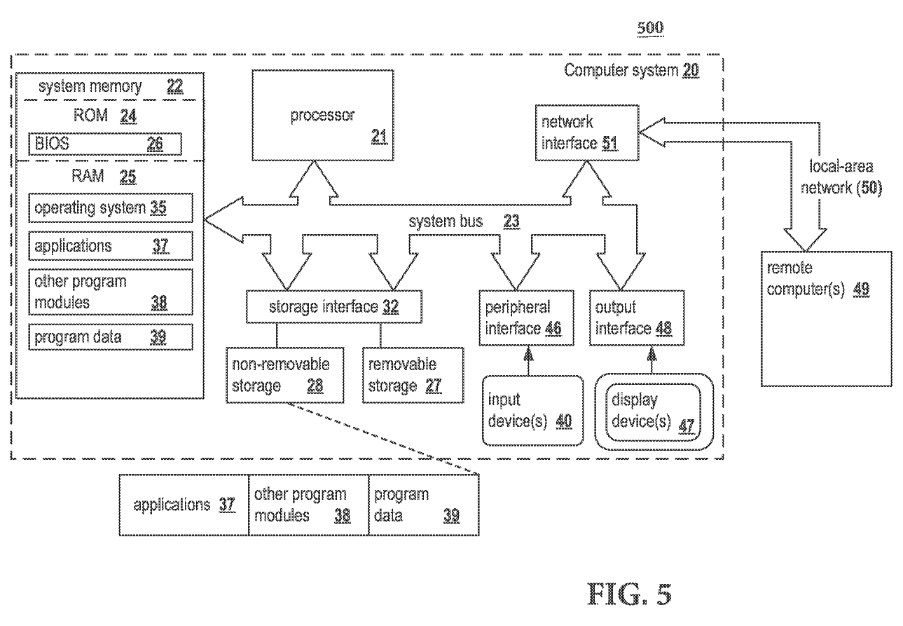

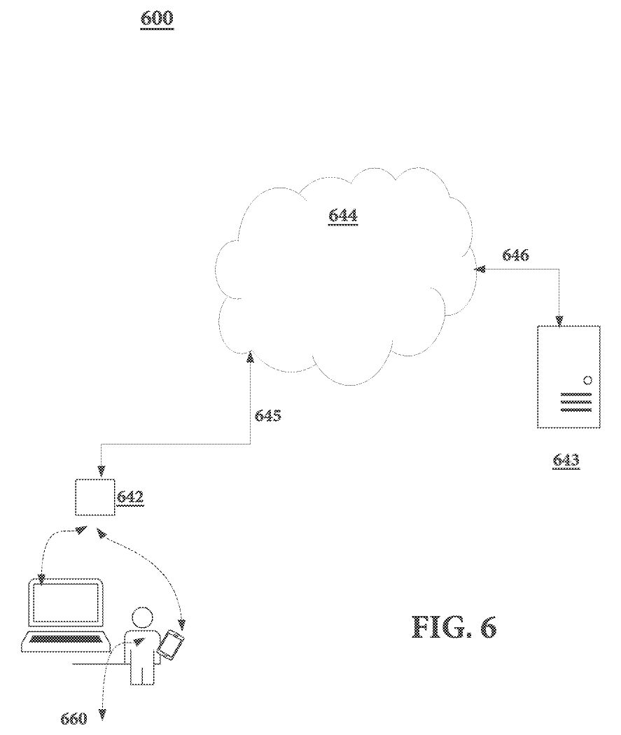

In step 480, method 400 includes transmitting the generated message for replenishment of the bulky product to an automated ordering system or staff. For example, the generated message may for replenishing product 121 may be sent either to an ordering system or staff member. The computing device maintains an association between each RFID tag identifier and a corresponding ESL address or identifier. Upon a valid placement event, the computing device resolves the ESL endpoint and attempts delivery of the display update over a network as described with respect to FIGS. 5-6. If the ESL is unreachable or a transmission error is detected, the computing device may queue the update for retry, generate an exception notification, and/or fall back to staff messaging while preserving an audit entry for later reconciliation.

In some implementations, the computing device writes an audit record including a timestamp, facility/location identifiers, product identifier, RFID tag identifier, detection confidence metrics (e.g., read count within the tag detection time period), any ESL update result codes, and the contents of any replenishment messages sent to staff and/or automated ordering systems. Audit records support traceability and reconciliation across inventory, replenishment, and display subsystems.

FIG. 5 is a block diagram 500 illustrating various components of an example computer system 20 via which aspects of the present disclosure may be implemented. The computer system 20 may, for example, be or include a computing system of the user device, or may comprise a separate computing device communicatively coupled to the user device, etc. In addition, the computer system 20 may be in the form of multiple computing devices, or in the form of a single computing device, including, for example, a mobile computing device, a cellular telephone, a smart phone, a desktop computer, a notebook computer, a laptop computer, a tablet computer, a server, a mainframe, an embedded device, and other forms of computing devices.

As shown in FIG. 5, the computer system 20 may include one or more central processing units (CPUs) 21, a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™, InfiniBand™, Serial ATA, I²C, and other suitable interconnects. The central processing unit 21 (also referred to as a processor) may include a single or multiple sets of processors having single or multiple cores. The processor 21 may execute one or more computer-executable lines of code implementing techniques in accordance with aspects of the present disclosure. The system memory 22 may be or include any memory for storing data used herein and/or computer programs that are executable via the processor 21. The system memory 22 may include volatile memory, such as a random access memory (RAM) 25 and non-volatile memory, such as a read only memory (ROM) 24, flash memory, etc., or any combination thereof. The basic input/output system (BIOS) 26 may store the basic procedures for transfer of information among elements of the computer system 20, such as those at the time of loading the operating system with the use of the ROM 24.

The computer system 20 may include one or more storage devices, such as one or more removable storage devices 27, one or more non-removable storage devices 28, or a combination thereof. The one or more removable storage devices 27 and non-removable storage devices 28 may be coupled to the system bus 23 via a storage interface 32. In an aspect, the storage devices and the corresponding computer-readable storage media may be or include power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory, such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology, such as in solid state drives (SSDs) or flash drives; magnetic cassettes, magnetic tape, and magnetic disk storage, such as in hard disk drives or floppy disks; optical storage, such as in compact disks (CD-ROM) or digital versatile disks (DVDs); and any other medium that may be used to store the desired data and that may be accessed via the computer system 20.

The system memory 22, removable storage devices 27, and/or non-removable storage devices 28 of the computer system 20 may be used to store an operating system 35, additional program applications 37, other program modules 38, and/or program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47, such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment as shown in FIG. 6, using a network connection to one or more remote computers 49. The remote computer (or computers) 49 may be or include local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks, such as a local-area computer network (LAN) 50, a wide-area computer network (WAN), an intranet, and the Internet. Examples of the network interface 51 may include an Ethernet interface, a Frame Relay interface, SONET interface, and wireless interfaces.

FIG. 6 is a block diagram of various example system components, usable in accordance with aspects of the present disclosure. FIG. 6 shows a communication system 600 usable in accordance with aspects of the present disclosure. The communication system 600 includes one or more accessors 660 (also referred to interchangeably herein as one or more “users”) and one or more terminals 642. In one aspect, data for use in accordance with aspects of the present disclosure may, for example, be input and/or accessed by accessors 660 via terminals 642, such as personal computers (PCs), minicomputers, mainframe computers, microcomputers, telephonic devices, or wireless devices, such as personal digital assistants (“PDAs”), smart phones, or other hand-held wireless devices coupled to a server 643, such as a PC, minicomputer, mainframe computer, microcomputer, or other device having a processor and a repository for data and/or connection to a repository for data, via, for example, a network 644, such as the Internet or an intranet, and couplings 645, 646. In one aspect, various features of the method may be performed in accordance with a command received from another device via a coupling 645, 646. The couplings 6 45, 646 may include, for example, wired, wireless, or fiberoptic links. In another variation, various features of the method and system in accordance with aspects of the present disclosure may operate in a stand-alone environment, such as on a single terminal. In one aspect, the server 643 may be a remote computer 49, as shown in FIG. 5, a computing device 110, as shown in FIG. 1, or a local server. In some implementations, communications between the computing device and ESL infrastructure employ authenticated and encrypted channels, and the computing device enforces authorization policies that restrict which users or processes may initiate replenishment and display updates.FIG. 7A is a schematic illustration of an additional implementation of a card B 700 configured for use within an inventory management and replenishment system. In the depicted implementation, card B 700 includes an RFID tag 702, which may be passive or active and is configured to be read by a reader-antenna when the card is removed from a holder and presented for scanning. RFID tag 702 is analogous to RFID tag 160B described elsewhere herein and may store an identifier and/or data associated with an item, bin, location, par level, or other inventory parameter. In certain implementations, card B 700 further includes a pull tab portion sized and shaped to facilitate manual removal and insertion of the card relative to a slot of a holder or housing. In some implementations, the pull tab includes a through-hole formed therein, the hole being sized to receive a tether, lanyard, cable, or ring suitable for physically retaining the card to a housing or fixture. The tethering feature may reduce misplacement or loss of the card during transitions between removal from a holder, presentation to a reader-antenna, and return to the holder, while permitting sufficient range of motion for reader engagement. For purposes of the disclosure, card A may be considered as implementing the same or similar features with respect to card B, described above.

FIG. 7B is a schematic illustration of an electronic shelf label bracket 710 configured to operatively associate an electronic shelf label with the housing and card system described herein. An electronic shelf label comprises a wireless, network-addressable display device configured to receive content updates over a network and to present visible indicia corresponding to item information, status, and/or par-level changes. In certain implementations, the electronic shelf label may be a commercially available, off-the-shelf Wi-Fi display device. In use, when a pull card (e.g., card B 700) is read by a reader-antenna, a computing device that receives the tag read communicates, via one or more intermediary servers over a network, corresponding content to be displayed by electronic shelf label. The electronic shelf label may be powered by an internal battery or external source, and may be mechanically retained within or upon a housing using bracket 710 as described with respect to FIG. 7C.

FIG. 7C is a schematic illustration of a housing module 720 configured to receive one or more cards and, in certain implementations, to support mounting of an electronic shelf label. Housing module 720 includes slot 722 and slot 724, which are analogous to slot 260A and slot 260B described in connection with earlier figures. Slots 722 and 724 are dimensioned to slidably receive corresponding cards, such as card B 700, in a side-by-side or stacked arrangement to support workflows involving multiple cards for a single item or location. In some implementations, housing module 720 further includes a front face configured to receive a label (e.g., a paper label), and/or a receptacle, recess, or mounting interface for an electronic shelf label, for example using bracket 710 in FIG. 7B. In some implementations, the housing may be identified, for example, as part number 51-0131-R, and a variant or associated part, for example, 51-0146-R, is configured specifically to hold an electronic shelf label such that the label is viewable from a front-facing position.

In some implementations, the housing module 720 is arranged such that cards are oriented in opposing directions, producing a visual indication of inventory status based on the number of visible pull tabs (e.g., two visible pull tabs indicating full, one indicating partial depletion, and none indicating depletion). This visual cue may supplement or operate independently of electronic detection.

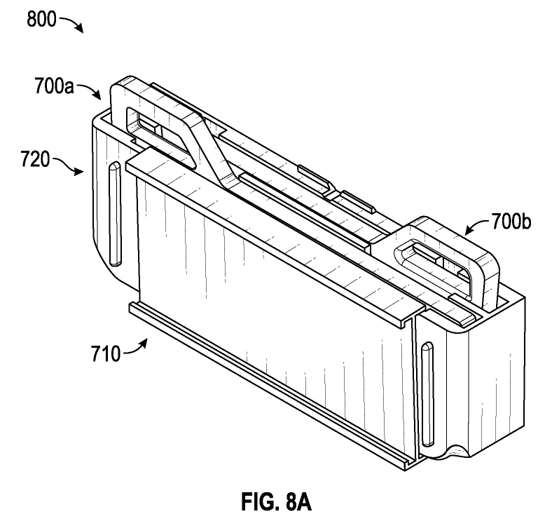

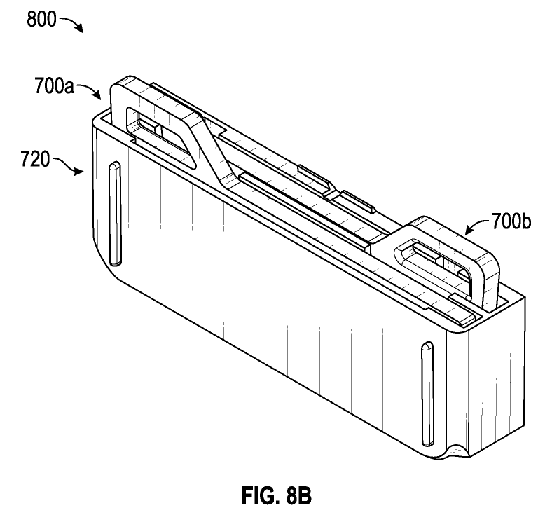

FIG. 8A is a schematic illustration of an assembled implementation including housing module 720 and electronic shelf label bracket 710, with two cards (card B 700a and card B 700b, each version of card B 700) respectively received within slots 722 and 724 of housing module 720 (analogous to slots 260A and 260B described elsewhere). In the depicted arrangement, the electronic shelf label bracket 710 is mounted to, received within, or otherwise supported by the housing, which in certain implementations corresponds, for example, to part no. 51-0146-R. In some implementations, the electronic shelf label is configured to display updated information, such as an adjusted par level or replenishment status, following a read of one of the cards by a reader-antenna. The system architecture enables the reader-antenna to detect the RFID tag on the pulled card, forward the read to a computer or controller, and prompt content changes that are communicated over a network (e.g., via one or more intermediary servers) to the electronic shelf label for display over a wireless (e.g., Wi-Fi) connection. In some implementations, the pull tabs of card A 700a and card B 700b include through-holes through which a tether, cable, or ring passes, thereby tethering the cards to the housing to prevent removal from the system or loss during movement from the holder to the reader and back.

FIG. 8B is a schematic illustration of an assembled implementation including housing module 720 with two cards (card A 700a and card B 700b) respectively received within slots 722 and 724 (analogous to slots 260A and 260B), and without electronic shelf label bracket 710 present. In this configuration, the housing’s front face (for example, part no. 51-0131-R) is configured to receive a paper label affixed or disposed on the front face to present item information and/or par-level information in lieu of an electronic display. As with FIG. 8A, the cards may include pull tabs with through-holes permitting tethering to the housing such that the cards remain retained within the system throughout removal, reading, and return operations. The underlying reader-antenna and computing workflow for card reads may be the same as described above, with the distinction that display updates are provided via the applied paper label rather than an electronic shelf label.

In various implementations, the methods described with respect to FIGS. 4-6 (e.g., detecting card removal and presentation, reading an RFID tag, updating inventory/par levels, generating replenishment signals, and/or logging transactions) may be carried out using and/or in conjunction with any of the components, modules, and assemblies depicted in FIGS. 7A-8B. For example, the methods may be performed using card B 700 (including a tethered pull tab with a through-hole), within housing module 720 having slots 722 and 724 (analogous to slots 260A and 260B), and optionally with electronic shelf label bracket 710 such that method outputs trigger corresponding ESL display updates via a networked communication path (e.g., via one or more intermediary servers to a Wi-Fi display). In implementations without an electronic shelf label, the same methods may utilize a paper label on the front face of the housing. These combinations are non-limiting, and the steps of FIGS. 4-6 may be executed mutatis mutandis using the foregoing hardware alternatives.

Features described in connection with FIGS. 7A-8B (e.g., the tethering hole, electronic shelf label mounting, housing variants, and slot arrangements) are presented as additional, non-limiting implementations and may be used individually or in any operative combination with the implementations described elsewhere herein.

Aspects of the present disclosure may be or include a system, a method, and/or a computer program product. The computer program product may include a computer readable storage medium (or media) having computer readable program instructions thereon for causing a processor to carry out aspects of the present disclosure.

The computer readable storage medium may be or include a tangible device that may retain and store program code in the form of instructions or data structures that may be accessed via a processor of a computing device, such as the computing system 20. The computer readable storage medium may be or include an electronic storage device, a magnetic storage device, an optical storage device, an electromagnetic storage device, a semiconductor storage device, or any suitable combination thereof. By way of example, such computer-readable storage medium may comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device, such as punch-cards or raised structures in a groove having instructions recorded thereon. As used herein, a computer readable storage medium is not to be construed as being or only being transitory signals per se, such as radio waves or other freely propagating electromagnetic waves, electromagnetic waves propagating through a waveguide or transmission media, or electrical signals transmitted through a wire.

Computer readable program instructions described herein may be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations in accordance with aspects of the present disclosure may be or include assembly instructions, instruction-set-architecture (ISA) instructions, machine instructions, machine dependent instructions, microcode, firmware instructions, state-setting data, or either source code or object code written in any combination of one or more programming languages, including an object oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be coupled to the user's computer via any suitable type of network, including a LAN or WAN, or the connection may be made to an external computer (for example, through the Internet). In some aspects, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform various functions in accordance with aspects of the present disclosure.

In various aspects, the systems and methods described in the present disclosure may

be addressed in terms of modules. The term "module" as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module’s functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system (such as the one described in greater detail in FIG. 5, above). Accordingly, each module may be realized in a variety of suitable configurations, and should not be limited to any particular implementation shown or described as an example herein.

In the interest of clarity, not all of the routine features of the aspects are disclosed herein. It will be appreciated that in the development of any actual implementation of features in accordance with aspects of the present disclosure, numerous implementation-specific decisions may be made in order to achieve the developer’s specific goals, and these specific goals may vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of various features in accordance with aspects of the present specification are to be interpreted by one of ordinary skill in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it will be apparent to those skilled in the art having the benefit of this disclosure that many more modifications than mentioned above are possible without departing from the innovative concepts disclosed herein.

What is claimed is:

1. An apparatus for managing an inventory of bulky products stored on a shelving system, the apparatus comprising:
an RFID antenna disposed at a designated location on a shelving system suitable for storing the bulky products, the RFID antenna configured to communicate with RFID tags disposed proximate to the RFID antenna;
an RFID reader including the RFID antenna, the RFID reader configured to communicate with at least one computing device, the RFID reader configured to control the antenna to scan and detect RFID tags disposed at the designated location on the shelving system, and to transmit messages to the at least one computing device, the messages indicating detections of RFID tags; and
a housing module affixed to a storage fixture of the shelving system on which bulky products are stored, the housing module being for storing a pair of RFID tagged cards, each RFID tagged card having a distinct respective RFID tag, the pair of RFID tagged cards in a same housing module being associated with a single bulky product, and a PAR level being established for the bulky product,
wherein the at least one computing device is configured to:
receive messages from the RFID reader indicating detections of RFID tags when the RFID tagged cards are placed at the designated location on the shelving system;
for each detected RFID tag, track a number of times the RFID tag is read during each tag detection time period, deduplicate received messages when applicable, and
when the number of times the RFID tag is read during the tag detection time period is at least two, generate a message for replenishing the bulky product associated with the RFID tagged card, wherein a quantity of the replenishment is based on the PAR level established for the bulky product.

2. The apparatus of claim 1, the RFID tagged cards including human readable labels or machine readable barcodes.

3. The apparatus of claim 1, wherein the PAR level corresponds to a predetermined quantity of the bulky product that corresponds to a single pair of RFID tagged cards.

4. The apparatus of claim 1, wherein the pair of RFID cards are individually removable from the housing module.

5. The apparatus of claim 1, wherein the pair of RFID tagged cards placed in a same housing module are interchangeable in that they are insertable in either of two slots available per housing module.

6. The apparatus of claim 1, wherein a shape of the RFID tagged card corresponds to a shape of a slot of the housing module such that the tagged card can only be inserted in a single orientation into the slot of the housing module.

7. The apparatus of claim 1, wherein the at least one computing device comprises a non-transitory computer-readable medium storing instructions that, when executed, cause the at least one computing device to perform the receiving, tracking, deduplicating, and generating steps recited in claim 1.7. The apparatus of claim 1, wherein the at least one computing device comprises a non-transitory computer-readable medium storing instructions that, when executed, cause the at least one computing device to perform the receiving, tracking, deduplicating, and generating steps recited in claim 1.

8. The apparatus of claim 1, wherein the deduplicating includes consolidating multiple detections of the same RFID tag within a tag detection time period.

9. A method for managing an inventory of bulky products stored on a shelving system suitable for storing the bulky products, the shelving system including an RFID antenna disposed adjacent a designated location and an RFID reader in communication with the RFID antenna and at least one computing device, the RFID reader configured to perform scans via the RFID antenna to detect RFID tags disposed at the designated location on the shelving system, the method comprising:
inserting a pair of RFID tagged cards in a housing module affixed to a storage fixture on which bulky products are stored, each RFID tagged card having a distinct respective RFID tag, the pair of RFID tagged cards in the housing module being associated with a single bulky product, and a PAR level being established for the bulky product;
when a predetermined quantity of the bulky product is removed from the shelving system, placing an RFID tagged card associated with the bulky product at the designated location on the shelving system, the predetermined quantity being a proportion based on the PAR level;
by the RFID reader, performing a scan to detect the RFID tag disposed at the designated location, and transmitting a message to the at least one computing device indicating detection of the RFID tag;
by the at least one computing device, receiving the message from the RFID reader indicating detection of the RFID tag placed at the designated location, tracking a number of times the RFID tag is read during each tag detection time period, deduplicating received messages for the detected RFID tag when applicable, when the number of times the detected RFID tag is read during the tag detection time period is at least two, generating a message for replenishing the bulky product associated with the RFID tagged card based on the PAR level; and
transmitting the generated message for replenishment of the bulky product to an automated ordering system or staff.

10. The method of claim 9, wherein each RFID tagged card includes a human readable label or a machine readable barcode.

11. The method of claim 9, wherein the PAR level corresponds to a predetermined quantity of the bulky product that corresponds to the pair of RFID tagged cards.

12. The method of claim 9, wherein the pair of RFID tagged cards are individually removable from the housing module.

13. The method of claim 9, wherein the pair of RFID tagged cards placed in the housing module are interchangeable in that they are insertable in either of two slots available per housing module.

14. The method of claim 9, wherein a shape of each RFID tagged card corresponds to a shape of a slot of the housing module such that the RFID tagged card can only be inserted in a single orientation into the slot of the housing module.

15. A computer-implemented system is provided for managing inventory of bulky products stored on a shelving system, the shelving system including an RFID antenna disposed adjacent a designated location and an RFID reader in communication with the RFID antenna and at least one computing device, the RFID reader configured to perform scans via the RFID antenna to detect RFID tags disposed at the designated location on the shelving system, the computer-implemented system comprising:
at least one memory; and
at least one hardware processor coupled with the at least one memory and configured, individually or in combination, to:
control the RFID reader to perform a scan to detect at least one RFID tag disposed at the designated location, wherein the at least one RFID tag is from a pair of RFID tagged cards stored in a housing module affixed to a storage fixture of the shelving system on which bulky products are stored, with each RFID tagged card having a distinct respective RFID tag, the pair of RFID tagged cards in the housing module being associated with a single bulky product, and a PAR level being established for the bulky product, wherein when a predetermined quantity of the bulky product is removed from the shelving system, an RFID tagged card associated with the bulky product is placed at the designated location on the shelving system, the predetermined quantity being a proportion based on the PAR level;
control the RFID reader to transmit a message to the at least one computing device indicating detection of the RFID tag;
by the at least one computing device, receive the message from the RFID reader indicating detection of the RFID tag placed at the designated location, track a number of times the RFID tag is read during each tag detection time period, deduplicate received messages for the detected RFID tag when applicable, when the number of times the detected RFID tag is read during the tag detection time period is at least two, generate a message for replenishing the bulky product associated with the RFID tagged card based on the PAR level; and
transmit the generated message for replenishment of the bulky product to an automated ordering system or staff.

16. The computer-implemented system of claim 15, wherein each RFID tagged card includes a human readable label or a machine readable barcode.

17. The computer-implemented system of claim 15, wherein the PAR level corresponds to a predetermined quantity of the bulky product that corresponds to the pair of RFID tagged cards.

18. The computer-implemented system of claim 15, wherein the pair of RFID tagged cards are individually removable from the housing module.

19. The computer-implemented system of claim 15, wherein the pair of RFID tagged cards placed in the housing module are interchangeable in that they are insertable in either of two slots available per housing module.

20. The computer-implemented system of claim 15, wherein a shape of each RFID tagged card corresponds to a shape of a slot of the housing module such that the RFID tagged card can only be inserted in a single orientation into the slot of the housing module.
